(19) United States
(12) Patent Application Publication
(10) Pub. No.: US 2007/0092873 A1
(43) Pub. Date: Apr. 26, 2007

(54) Digital identification of genetic materials and methods for acquiring data for it

(76) Inventors: Alexey Khanifovich Baymiev, Ufa (RU); Alexey Viktorovich Chemeris, Ufa (RU); Dmitry Alexeevich Chemeris, Ufa (RU); Timo Kalevi Korpela, Turku (FI); Nikolai Glebovich Usanov, Ufa (RU); Vener Absatarovich Vakhitov, Ufa (RU)

Correspondence Address: PAUL & BARBARA GENTRY, 13131 SNUG HARBOR ROAD, BILOXI, MS, 39532, US

(21) Appl. No.: 10/503,410

(22) PCT Filed: Jul. 2, 2003
(86) PCT No.: PCT/EP02/07854

§ 371 Date: Jul. 30, 2004

(30) Foreign Application Priority Data
Jul. 13, 2001 (US) 09/905491

(51) Int. Cl. C12Q 1/68; G06F 19/00

(52) U.S. Cl. 435/6; 702/20

(57) ABSTRACT

The present invention relates to methods using HIPK1 sequences for use in diagnosis and treatment of lymphoma and leukemia. In, addition, the present invention describes the use of these compositions for use in screening methods.

This is national stage application under 35 U.S.C. section 371 of international application WO 03/066899 filed on Feb. 7^{th d} 2003 and published on Aug. 14th 2003, said international application claiming priority of the Finnish national patent application FI 20020260 filed on Feb. 8th 2002.

### FIELD OF INVENTION

The present invention provides methods for obtaining and presentation of genetic information of various organisms. In particular, the invention describes methods for rapid comparison of DNA sequences in digital form. The invention is commercially applicable in biotechnology, medicine, criminology, food technology, and other fields of human activities where the identification systems to characterize prokaryotic and eukaryotic organisms are needed.

### BACKGROUND OF THE INVENTION

“Artificial unification” of microbial groups for their biochemical and morphological similarity is a well-known method in taxonomy. The propinquity of testing strains is established in accordance to a large number of laboratory tests and analyses [1]. However, this method is laborious, expensive, and takes a long time, but cannot give well-defined answer about the propinquity of two related strains or genetic position of a single undefined microbial culture. It cannot also reveal objective phylogenetical differences between strains of genetically close microorganisms. The computerized presentation of the results achieved by this method is complicated, especially in data-base form and can be performed only as a text collection of different parameters. It is inconvenient, ambiguous, and needs a large amount of memory space in a computer. The results of a computer search and comparing of different strains cannot be conveniently sent through Internet without special programs.

The principle of so-called “numerical taxonomy” is more objective than the artificial unification [2]. In numeric taxonomy all microbial criteria can be taken into account, if measurable parameters can have distinctively different meanings and can be expressed in the form of “+” and “−” and thereafter be subjected to a computer analysis. The coefficient of similarity can be then calculated according to the equation (1):

$$S = \frac{M + P}{M + N + P + Q} \qquad ( equation 1 )$$

wherein,

M and P are the sum of properties, which are the same for both strains of microorganisms A and B (M is a positive reaction and P is a negative reaction), N is the sum of properties, which are positive for A and negative for B strains of microorganisms, Q is the sum of properties which are negative for strain A and positive for strain B. For a value of S=1, it can be assumed that strains A and B are the same or near-identical, but if S<0.02 the strains are considered different.

The method of numerical taxonomy is simple in the presentation as to the identity of organisms, but still it has a serious drawback of being very laborious, demanding an excessive range of diversified analytical techniques producing high costs in the form of work, reagents, and equipment. This method also requires efforts to get proper computer description of the testing results and the results need a large volume of computer memory being inconvenient for the work through the Internet.

The method of microbial specification based on the determination of DNA sequences coding highly conservative genes of microbial ribosomal 16S RNA (mainly it relates to prokaryotes) is widely accepted [3]. Presently, this method is being used for phylogenetical analysis of unidentified bacterial strains. It is assumed, that the method allows to prove the direct phylogenetical position of a studied bacterial strain. The results of such study can be easily transformed into computer-acceptable digital form as the text DNA sequences coding corresponding gene of 16S RNA. These texts of sequences do not need big volume of computer memory space (only 1600-1800 bytes). The results received in different laboratories are reproducible.

The most serious disadvantages of the 16S RNA method relates to its high costs and long analysis time, usually taking a few days. Moreover, it is limited only to prokaryotic microorganisms. The differences in the primary structures of 16S RNAs of different prokaryotic organisms reflect only the divergence of certain conservative genes, but not the differences in the whole bacterial genomes. In particular, the method cannot evidence the horizontal gene transfer and other fast genetic processes observed among prokaryotes. A difference of 5-10 bp in a 1500 bp sequence of two bacterial strains classifies these microorganisms as belonging to the same species. Hence, 16S RNA method cannot be unambiguously employed for differentiation of subspecies or serotypes of bacteria. Such a differentiation is especially important for protection of intellectual property rights for microbial producer strains in biotechnology.

Certain molecular biology techniques provide possibilities of revealing DNA variability of all organisms, including eukaryotes. Restriction endonucleases in combination with Southern blotting with corresponding probes allow detection of the restriction-fragment-length polymorphism (RFLP). Highly polymorphic loci consisting of short tandem repeats are often used in blotting experiments [4-5] as probes. Such repeats were found in genomes of many organisms; one of them, the gene for protein III of the M13 single-stranded phage, is used in such studies [6-8]. In addition to M13, other minisatellite repeats of various origin were used in blotting experiments in order to reveal RFLP [9]. However, these studies are highly expensive, laborious, and need personnel of the highest qualifications. Results of such studies yield only empirical characterisation and cannot give explanations outgoing from genomic structure. Further, the results received in different laboratories are often irreproducible due to many uncontrollable parameters. The results cannot be expressed in short form convenient for computer analysis.

Randomly amplified polymorphic DNA (RAPD) analysis is also presently used for the classification of DNA sequences. It exploits polymerase chain reaction (PCR) with various “arbitrary” primers. Modifications of this method (DAF, SSP, AFLP, IMA, and RAPD-RFLP) are used for special purposes [10]. All these methods have the same disadvantages as RFLP.

An approach is based on digestion of total DNAs of microorganisms with highly specific restriction endonuclease-enzymes and separation of the reaction products with the aid of pulse-field gel-electrophoresis (PFGE). The peculiarity of this method is the separation of a rather small number of high-molecular-weight DNA fragments (usually varying from 10 to 800 kb) with an apparatus specially designed for such experiments. The electrophoretic patterns of microbial DNAs consist of a number of bands characteristic of each strain. However, a drawback is that determination of the exact size of an oligonucleotide in any of the bands is impossible. Moreover, in the PFGE patterns, the distance between bands strongly depends on the conditions of electrophoretic separation (quality of chemicals, type of apparatus, electric field, size of DNA fragment, etc.). Interlaboratory reproducibility is poor, and thus this approach is not suitable for digital identification of bacterial strains [11].

Recently, a new technique, restriction fragment end labelling (RFEL)[12], was worked out. It allowed discrimination of closely related microbial strains of Rizobium galegae with high sensitivity [13]. For the analysis of Rizobium galegae strain polymorphism, bacterial DNA was cleaved with the endonuclease HindIll. After end labelling with [P³²] dATP, restriction fragments were separated by high-voltage electrophoresis in denaturing conditions. The position of 60 bands on the radioautograph were taken into account to get distinguishing information between different Rizobium strains. Authors suggested to use the images of radioauthographs received in the presence of standard arbitrary blank primers (having known length of oligonucleotides) for description and discrimination of different microbial strains belonging to group of Rizobium galegae.

Despite of relatively high sensitivity of the above-described method, it brings about a number of drawbacks. First of all, the restriction endonuclease HindIII, offered for fragmenting Rizobium galegae DNA, cannot be used for many genera of prokaryotic and eukaryotic microorganisms, since the DNA fragments received with Hind III can be too few to get satisfactory distinguishing information between strains of prokaryotic microorganisms. On the other hand, this number can be too high especially with eukaryotic microorganisms, such as yeasts and fungi. The main drawback of RFEL, however, origins from an unsuccessful choice of data presentation in the form of photographic images of autoradiographically developed electrophoregrams. Such photographs contain 30-50 line patterns and are very inconvenient for subsequent processing. In particular, comparison and interpretation of two or more images is difficult. In addition, photographic images have an analogous mode of presentation of information and this fact necessitates a large volume of computer memory. For example, to place the information characterising the DNA structure only of one bacterial strain (in form of black and white 256 bit *.gif image format prepared by scanning of radioautograph), requires more than 50-100 Kb of memory space. The data cannot easily be mailed or transferred through Internet. Complicated and expensive image-recognizing computer programs are needed for automation of the development and characterizing work.

The present invention avoids all the drawbacks of the above-described methods by utilizing selected restriction endonuclease (s) for digestion of whole DNA of an organism and thus produces a significant improvement over the prior art. The obtained fragments are analysed according to their size and the results arranged in a form allowing a convenient digital presentation.

### BRIEF DESCRIPTION OF DRAWINGS

FIG. 1. The illustration (FIGS. 1A-C) demonstrates the model of restriction pattern received in silica for genomic DNA of Bacillus subtilis [16] cleaved by the restrictase PciI (length of genome is 4,212,814 b.p, common number of cuttings—737) and different forms of its presentation. All data in FIG. 1 have been calculated by the method of computer imitation experiments performed with the program “Silicone tube v.2.4” FIG. 1A. Demonstrates the full analog form of data characterizing the restriction of DNA in silica. The distribution of oligonucleotides in accordance to their length (from 1 b.p. to 5000 b.p., axis X) is shown on the plot in form of vertical lines (bands). Axis Y demonstrates the number of different oligonucleotides in one band of separation pattern having the equal length.

FIG. 1B. Short analog form of data presentation received on the basis of FIG. 1A. The number of oligonucleotides in one pattern (axis Y) was not taken into account for rendering of an insufficient information. The longest part of oligonucleotides (more than 576 b.p.) is discarded. Bands of oligonucleotides (having length less than 32 b.p and more than 544 b.p. as emphasized in FIG. 1-B by grey colour) are not included into DC. FIG. 1C. Digital form of presentation of a restriction pattern of genomic DNA of Bacillus subtilis cleaved in silica by PciI. The 512-bite computer calculated DC of B. subtilis [16] in hexadecimal presentation was calculated by the analog data from FIG. 1A.

FIGS. 2(A-C). As in the FIG. 1, but for genomic DNA of Escherichia coli 0157:H [17] cleaved by Mfe I (hex). The length of genome is 5,529,376 b.p, common number of cuttings per whole DNA molecule is 1412.

FIGS. 3(A-C). The same as in FIG. 1, but for genomic DNA of Neisseria meningitidis MC58 [25] cleaved by Xma III (hex). The length of genome is 2,272,351 b.p, and common number of cuttings per whole DNA molecule is 723.

FIG. 4. The same as in FIG. 1, but for genomic DNA of an Archeae bacterium Aeropyrum pernix KI [27] cleaved by Nhe I (hexa). The length of genome is 1,669,695 b.p, and common number of cuttings per whole DNA molecule is 729.

FIG. 5. The same as in FIG. 1, but for genomic DNA of a pathogenic bacterium Mycoplasma genitalium G37 [28] having one of the shortest bacterial genomes. DNA was cleaved by Nhe I (hexa). The length of genome is 580,074 b.p, and common number of cuttings per whole DNA molecule is 398.

FIG. 6. Calculation of DC of a short-genome strain Mycoplasma pneumoniae M129 [29] by fourth restrictases: AgeI, EcoRI, NheI, SpeI showing relatively rare character of restriction with this type of DNA. All data was obtained with computer simulation with program “Silicone tube v2.4”.

FIG. 6A. Computer calculated patterns of DNA cleaved with Age I.

FIG. 6B. Computer calculated patterns of DNA cleaved with Eco RI.

FIG. 6C. Computer calculated patterns of DNA cleaved with Nhe I.

FIG. 6D. Computer calculated patterns of DNA cleaved with Spe I.

FIG. 6E. Restriction pattern of joint action of four restrictases.

FIG. 6F. The 512-bite DC of M. pneumoniae M129 [29] in hexadecimal presentation obtained from analog data of FIG. 6E.

FIG. 7. Graphical illustration of genetic similarity of two strains Neisseria meningitidis of serotype A and B [25, 26] shown by their DCs. Axis X—the number of position of hexadecimal number (cluster) in DCs of each strains, axis Y—the decimal meaning of each hexadecimal number (cluster) in Digital Code for both strains. Straight line is N. meningitidis MC58 serogroup B and dotted line belongs to N. meningitidis Z2491 serogroup A.

### DETAILED DESCRIPTION OF INVENTION

The present invention relates to the field of classification and identification of genetic material. Information on primary sequence data of nucleotide sequences of DNA and RNA is nowadays increasingly accumulating along with the intensive studies being made in molecular biology providing a commercially increasingly important field. Especially, large genome projects for sequencing whole genomes of different organisms, including man with automated processes, create needs to analyse the sequences for various purposes. It is obvious that commercial test kits including reagents, instructions, soft ware etc. will be available. Such a market can be forecasted to increase rapidly. The present invention also relates to the methods for obtaining of compact digital data characterizing the primary structure and the length of DNA of individual organisms, which data can be conveniently used in computer applications.The present invention can be exploited in revealing DNA similarity or variability in prokaryotic as well as in eukaryotic organisms. In particular, the invention relates to the modern techniques of genetic identification of various microorganisms. The described procedures can be used for the determination of taxonomical position of the microbial organisms or propinquity of related microbial strains, which are of a key importance for legal protection of microbial producer strains, donors of new genes and so on.

The laboratory work of the present invention involves extraction and purification of total DNA from an organism under concern. In certain cases, DNA fraction may be divided into subfractions, like chromosomes, plasmids, etc., before the next step, which is its digestion by restriction endonucleases (restrictases), to get a more clear picture for a certain DNA or RNA fraction. The digestion mixture should contain relatively short oligonucleotide fragments, because they must be separated according to their lengths (sizes) usually expressed by the number of their nucleotide bases. After the separation, whether the restriction endonucleases are correctly selected, the resulting fragments do not form a continuous series of fragments distributed according to their length, but form specific patterns. This knowledge of the pattern of different sizes of oligonucleotides contains information, which after due rearrangements and analysis, according to the present invention, allows one to deduce relationships and origins of the genetic material. The required laboratory techniques for obtaining said data in analog form of separation patterns (pictures, photos) are known in theory in the prior art. The essence of the laboratory methods are found e.g. in the handbook by Maniatis, T., et al. [ref. 14].

According to the present invention, comparison and manipulations of the information, received in analog form of separation patterns, can be conveniently made after transforming this data into a compact hexadecimal digital form (Digital Codes, abbreviated here as DCs) bringing about data characterizing the DNA length and structure.

The main difficulty for achieving the final objectives of the present invention, i.e. presenting the data in a convenient digital form, expressly lies in a proper selection of restrictases and separation methods in the context of the origin of the DNA material. This means that certain restrictases are preferable to certain DNAs.

DCs are obtained from the analog data of the separation patterns by determining the presence or absence of each possible oligonucleotide from the DNA digest in 3 alternative discreet ranges of sizes (number of nucleotide bases in an oligonucleotide). These ranges are tentative only, because basically in the same way other size ranges can be employed. However, for practical applications, in particular for organizing this data in the form feasible for computers, a standardization of the ranges and methods, in general, is preferable. Therefore, the present invention focuses to describing the most useful model, optimal to the present practical limits of separation techniques. In the future, however, when these laboratory techniques may be improved, the optimal methods may change accordingly. Restrictases of wide specificity ranges exist; some of them can split DNA to nucleotide levels, but in the same time other restrictases can split a large DNA only from a few points only. The enzymes can be isolated from a number of sources as extremely pure preparations. They are available from many commercial sources at a relatively low price, since restrictases have been long exploited in routine molecular biology research. The present invention is mainly based on restrictases recognizing relatively many but not the highest number of oligonucleotides. The preferred recognized sequence size is 4-8 nucleotides and includes the direction of the sequence (upstream or downstream).

The specificities (the length of recognized nucleotide sequences and inherent specificity to certain sequences) of commercially sold restrictases are practically always known and the selection of restrictases is not totally experimental approach.

The knowledge of the specificity and purity of a restrictase is of key importance for the present invention, because otherwise reproducibility of the results in different laboratories will not be optimal. In the case of an impure or low-specificity enzyme, the molecular significance of the results is blurred, even though they still can reflect for a certain relationship or similarity of organisms. The election of a restrictase employed for the hydrolysis of a DNA is critical because it must digest DNA into pieces of optimal lengths, which can be separated in practise according to their sizes by available methods and equipment.

Available separation techniques are limited to a range from tens to hundreds of oligonucleotides [15]. Very high specificity with specific recognition of a long sequence will cut DNA to too long pieces, which cannot be separated and, correspondingly, the total information will be poor. These long pieces cannot be separated according to their length with high accuracy (i.e. about 1 b.p.) and due to this reason the digitalization will be also hampered.

In certain cases more than one restrictase of low cutting-frequency can be applied to get oligonucleotides of suitable compositions of sizes. Different restrictases producing an optimal composition of a mixture of oligonucleotides may be required for different classes of organisms, exemplified by viruses, bacteria, yeasts, fuingi, plants, and animals.

Whole DNA of even bacterial origin is in a molecular level enormously long. One pure and highly specific restrictase do not split whole DNA into enough small pieces, but recognizes only certain specific sequences.

The basic embodiment of the present invention is that every cut reflects the presence of this specific sequence recognized by a certain selected restrictase. If two bacteria, for example, are related species, it is probable that there are more related patterns of restriction, the length of genome, and also more related sizes of oligonucleotides in the digestion mixture of the total DNA. If enough large material of oligonucleotides is analysed, probability of two species having equal oligonucleotide composition in the digest will approach zero. On the other hand, if two species are identical, they will always give equal oligonucleotide composition independent of how large material is analysed.

Whole DNA of a studied microorganism affects the results of a consequtive action of one or more of restrictases. They have preferable a hexanucleotide recognition site and it is possible to transform whole genomic DNA, independent of its length, to 50-3000 fragments of different sizes. The statistical requirements for obtaining an adequate information for a unique characterization of a DNA sequence was shown experimentally and by computer modelling to lie within this range giving an optimal data in the present invention.

Digestion products of a DNA, consisting of a mixture of the whole DNA fragments with different sizes (different number of bases in DNA chains, or lengths) are first separated according to their sizes. There are available a variety of electrophoretic and other methods are found in textbooks. The results of separation are conveniently described by organizing them in the form of a table having two or more horizontal rows (depending on the modification of the method) and 128 (or 256, or 512) vertical columns. The number of rows depends on the number of restrictases used. The uppermost row of the table can, for example, demonstrate the number of bases in corresponding pattern of separation in an order of growing from left (beginning) to right (to the end of counting). The first column corresponds to the oligonucleotide fragment with 33 nucleotides. This beginning can be any number, but the starting address #33 was chosen here due to the characteristics of usual separation techniques with oligonucleotides shorter than 30 b.p. Another reason for choosing the starting point of #33 is the fact that the most informative frames lie in the regions of oligonucleotide patterns having the relatively shortest length (more than 30 but less than 100 b.p.) which was demonstrated by computer modelling experiments. If needed, possible additional lower rows can be added in the same way to show the presence or absence of the corresponding size patterns of their separation products. These results are tabulated in the same way at the positions indicating the number nucleotides of the fragment. Such final table is formed of individual columns or boxes indicating if there exists an oligonucleotide of certain size within the product mixture or not.

If there is an oligonucleotide in a size pattern having the length of M bases in the restrictase digestion product, a digital significance of 1 enters the consequent box below this number. If a pattern having the length of N bases in the oligonucleotide chain is absents in the separated mixture, the digital meaning of 0 enters consequent box below this number. Binary numbers 0 or 1 must be filled in all boxes of the table starting from column #1 to the end of the row. The last meaning of the row (or column) number is 128, 256 or 512, depending on the chosen variant of the method.

The length of frame for digitizing is fixed and involves patterns of separation having 128, 256 or 512 of possible oligonucleotide fragments with differences in size in one base and present or absent in products of DNA digestion obtained by one restrictase. Number of columns (128, 256, or 512) in the table defines the length of binary numbers needed for its next transformation into hexadecimal digits.

The resulting binary numbers created after joining of neighboring cells in the rows into one “box” consist of 128, 256, or 512 digits having alternative discreet meanings 1 or 0 which must be transformed into the hexadecimal numbers. The last procedure can be done by needed calculations according to known algorithms. 32-Byte hexadecimal numbers will be created from 128 binary contents of “boxes”, 256-bites digit will give 64-byte hexadecimal “words”, 64-byte will be received if to use variant with 512 nucleotide fragments.

These unique numbers are very suitable for comparative computer and hand analyses of two or more organisms, for purposes of phylogenetic taxonomy, for fine identification of closely related strains relative on the level below than subspecies as illustrated in Examples. Hexadecimal form is the most compact for computer work and data base organization when comparing with other types of records such as decimal numbers or the form of letters as in usual words.

In common, DC of different organisms reflects the results of interaction process between DNA and Chosen Type of Restrictase (CTR) used for digestion. It means that the information characterizing CTR must be also presented simultaneously with DC to make this data exact. In other words, data characterizing DNA can be organized in the form of two hexadecimal digits (CRT+DC) like the binary coordinate characterizing the position of an organism “on the surface of biological diversity”.

CTR data can be also presented in different ways, for example, as trivial name like EcoRI, or digitally as usual decimal numbers, characterizing the position of concrete restrictase in specified and coordinated list of restrictases. From the point of computerization, it is more convenient to characterize the type of restrictase as a hexadecimal number.

The hexadecimal numbers characterizing the restrictases may be presented as their position in a list of commercially available restrictases. However, the list organized in such a manner will be beforehand limited and later discovered restrictases will not be available for the process of DC making. There are different possibilities for organizing data characterizing the type of restrictases in hexadecimal digital form not bringing the features of a normal register of records. As an example on such data organization can be taken the process of presentation for the restrictases having hexanucleotide palindrome of binding. The nature of such restrictases is determined by the site of binding marking like a word from 6 letters. Each letter may have only four meaning (AGCT) corresponding to different nucleotides. These “words” may look like “AAGGCT” or “GGTACC” etc. Restrictases with wide specificity (restrictases of strictly determined binding sites may be designated by letters N or P etc.) are not considered here, because they are not usually applicable in this invention. There are only 4096 combinations from starting “word” AAAAAA to finish TTTTTT, which can be expressed as a discontinuous row of 3-byte hexadecimal number from the 000 to FFF. The position of place of restriction inside of “word” can be also determined as fourth digit in CRT on the next manner: ↓XXXXXX-0(hex), X↓XXXXX-1(hex), . . . XXXXX↓X-6(hex), . . . XXXXXX-7(hex). The latter does not specify the case, while is needed for theoretical purposes.

According to the above discussion, the trivial name of restrictase EcoRI, for example, is unambiguously transformed into the hexadecimal number 83D2.

It is understood that the present invention describes only some variants of the basic embodiments of the invention. Different restrictases with different specificities can be applied to produce different sets of oligonucleotides. In all cases, the sizes of the oligonucleotides can be arranged so that they can be presented in a digital form. The method of tabulating them, as explained above, is only one simple, convenient, and illustrative approach. There are available other mathematical and computer methods to automate the digitalization process in the future. A simple computer program termed “Silicone tube” was worked out here for carrying out the manipulations in Examples 2-8. The algorithms of “Silicone tube” are evident for a person skilled in the art and the required information is directly derivable from the description of the invention.

The basic embodiments of the present invention are:

i) hydrolysis of DNA by selected restrictases,

ii) grouping of the product oligonucleotides according to their sizes,

iii) presenting this information in a digital form, and

iv) the exploitation of the digitalized data for identification of DNAs.

In particular, characteristic to the present invention differentiating it from the prior art is the following:
i) The main information for identifying a DNA is obtained from restriction map of oligonucletide chains. The lengths of the oligonucleotides are considered independently of their primary structures or other properties. The number of nucleotides in the oligonucleotides is determined with the accuracy of 1 nucleotide. The amounts of oligonucleotides are not measured quantitatively.
ii) Constant series of shorter oligonucleotides which are present in the digestion mixture (usually 1-10%) are considered. Fragments with lengths of more than 600 nucleotides are not usually considered.
iii) The first and last number of the constant series of oligonucleotides are fixed.
iv) One band in the restriction map involving more than one different oligonucleotides has the same information value as the one with one oligonucleotide species.
v) Absence of an oligonucleotide on restriction map (void place) has same informational value as real oligonucleotide. The former gets digital number 0 whereas the latter gets number 1.
vi) Usually the sum of all bands on oligonucletide map (imaginary or real) must be divisible with 16.
vii) The present invention is a combination of a number of individual previously know details which are logically combined together with inherent empirical rules.

The invention is further illustrated by specific non-limiting examples. Even though the examples describe mainly classification of certain prokaryotic organisms, this invention is equally well suitable to DNAs from any eukaryotic organism, including human DNA. Eukaryotic organisms are exemplified here by Saccharomyces cerevisiae [30-46].

### EXAMPLE 1

Demonstrates a laboratory method of digital characterization of total DNA from two Rizobium sp. strains. This Example describes in a condensed form all essential procedures and manipulations for achieving the digital codes starting from primary data. Because the labour needed to make such procedures manually is enormous, a simple computer program termed “Silicone tube” was worked out for carrying out the manipulations in Examples 2-8. The algorithms of “Silicone tube” are evident for a person skilled in the art and the required information is directly derivable from the description of the invention.

Two strains of Rhizobium sp., received from the Microbial Collection of the Institute of Biochemistry and Genetics (Russian Academy of Sciences, Ufa) and designated as R702 and R703, which have no differences in cultural and morphological characteristics, were cultivated on Petri dishes with nutrient agar medium for two days at 35° C. Total DNA was isolated from a bacterial colony containing approximately 10⁶ cells, after its preliminary treatment with lysozyme and sodium EDTA, with the standard sodium perchlorate—phenol—chloroform method described by Maniatis et al. [14]. Rhizobium DNA was cleaved with restriction endonucleases HindIII and EcoRI by an overnight reaction. Cohesive ends of the fragments were labelled with [α-³²P] dATP (2.5 μCi per reaction) with using the exo Klenow fragment of E. coli DNA polymerase I for 1 h at 37° C. The uni-incorporated label was removed by the ethanol precipitation. Samples were dissolved in 6 μl of TE (10 mM Tris-HCl, ImM EDTA, pH 8.0) and then 4 μl of stop solution were added (95% formamide, 20 mM EDTA, 0.05% bromphenol blue, and 0.05% xylenol blue ). For denaturation of DNA, samples were heated at 80-85° C. for 2 min immediately before loading of the preparation onto a sequencing gel. Electrophoresis was run on a 5 and 6% polyacrylamide gels (acrylamide:methylene bis-acrylamid 19:1) with 7 M urea at 55° C. in the presence of another arbitrary sample containing a mixture of oligonucleotides with defined length and labelled with [α-³²P] as described above.

After the electrophoresis, the gel was sequentially treated with 10% acetic acid and 10% ethanol to fix nucleic acids and to remove urea. Then the gel was dried at 80° C. and exposed to an X-ray film with routine methods. After the development of the film, the positions of the each restriction patterns of DNA were characterised on the autoradiograph by comparing with position of the corresponding pattern in the standard mixture. The results of measurement are collected in Table 1. On the basis of this experimental data, the digital codes (DC) of the strains R702 and R703 were determined.

Data provided 128-bite hexadecimal numbers:

For strain of Rhizobium sp. R702

a) in short 128 bite form:

1C.C6.52.1A.25.4E.74.A8.D2.9E.A2.24.70.B3.26.22 (128-byte DC obtained for the strain R702 with Hind III in hexadecimal form), or 28.90.9D.65.5A.E8.D3.0A.54.E9.5F.2E.BD.24.02.1F (128-byte DC obtained for the strain R702 with EcoRI in hexadecimal form).

b) in 256 byte form:

1C.C6.52.1A.25.4E.74.A8.D2.9E.A2.24.70.B3.26.22. 28.90.9D.65.5A.E8.D3.0A.54.E9.5F.2E.BD.24.02.1F (a combined 256-bytes DC of the strain R702 determined for both Hind III and EcoRI). The same for the strain R703:

c) in short 128-bites form:

04.80.4E.2D.64.A2.0E.01.A2.44.48.8C.90.4D.47.22 (with Hind III) 64.91.97.14.42.04.C4.91.1A.22.44.00.88.C8.12.26 (with EcoRI)

d) in 256 bite form:

04.80.4E.2D.64.A2.0E.01.A2.44.48.8C.90.4D.47.22. 64.91.97.14.42.04.C4.91.1A.22.44.00.88.C8.12.26 (with HindIII and EcoRI)

### EXAMPLE 2

The experimental techniques (isolation of DNA, hydrolysis and electrophoresis) are known procedures from prior art. The Examples 2-8, therefore prove the applicability of the invention for various purposes with using the available data from literature. It is illustrated that such digital codes or DNA-passport can be obtained also by using gene sequence information. It also shows that the sequence and restriction data are compatible. Without this, the present invention would have only a limited use. Undoubtedly the same primary sequence data as now taken from literature could have been obtained also by us. On these grounds, the results of Examples 2-8 were worked out in silica by computer simulation experiments.

Example 2 demonstrates the process of calculation of DCs for various types of genetic materials having differences in primary structure, and the length of DNA molecule. Various types of endonucleases were used for the restriction. Process of formalization (discarding of insufficient information) starting from analog form (FIGS. “A and “B) to compact hexadecimal digits (FIGS. “C”) were obtained with program “Silicone Tube 2.4”. The main characteristics of the bacterial genomes in computer simulations are presented in Table 2.

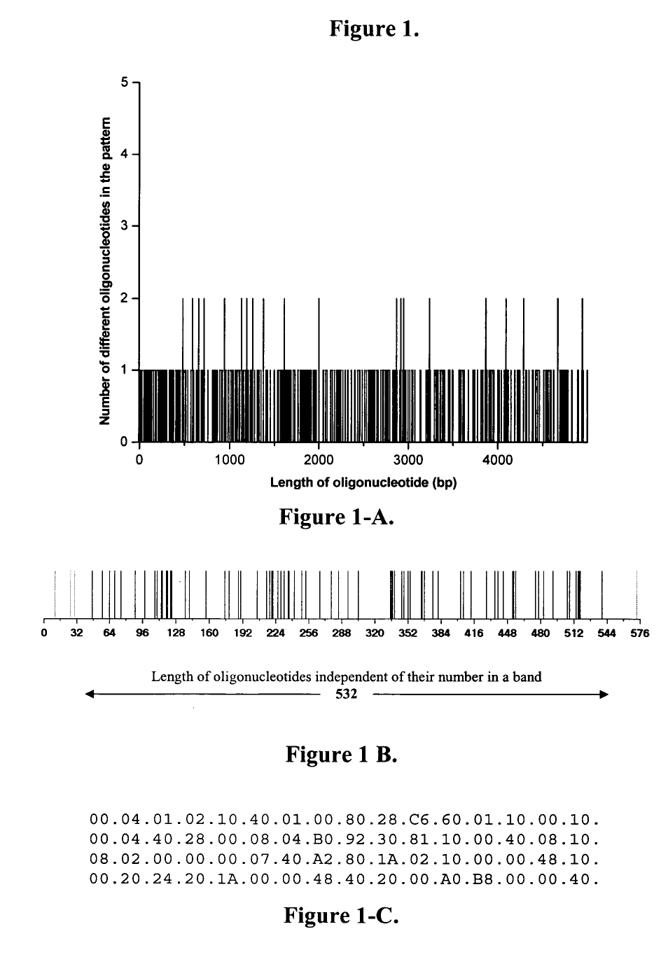

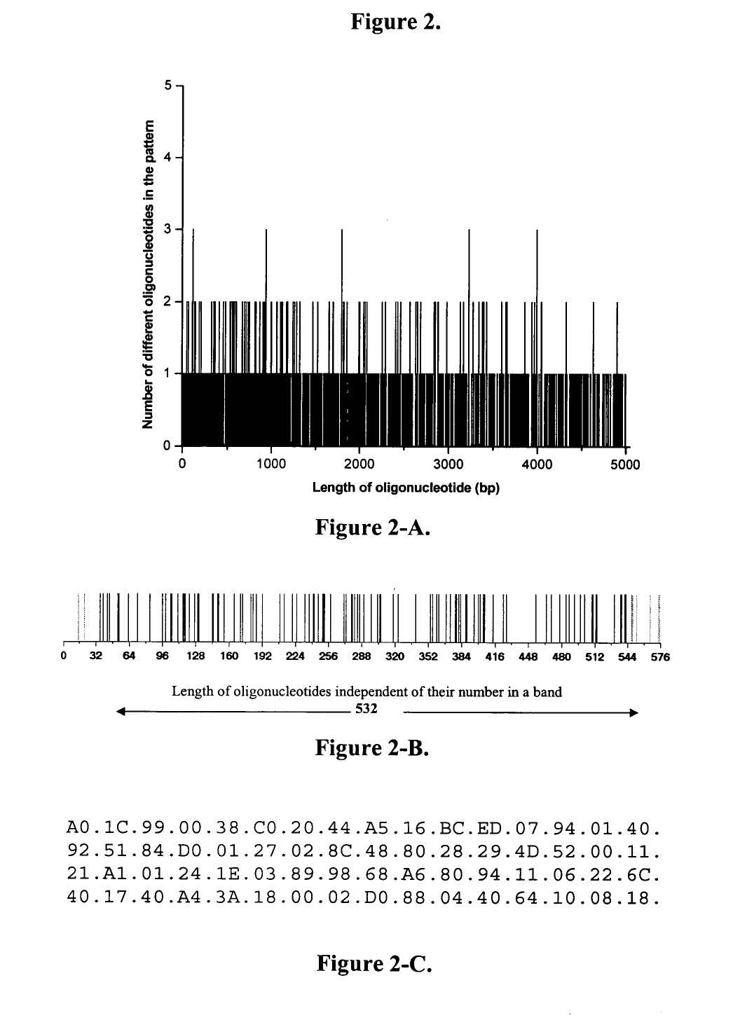

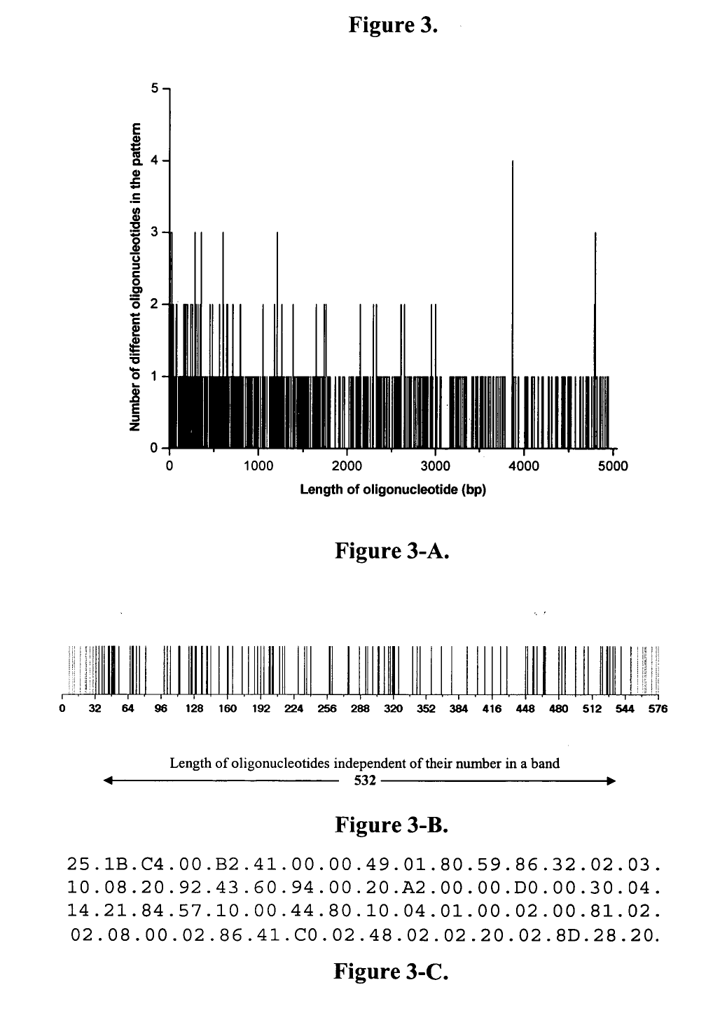

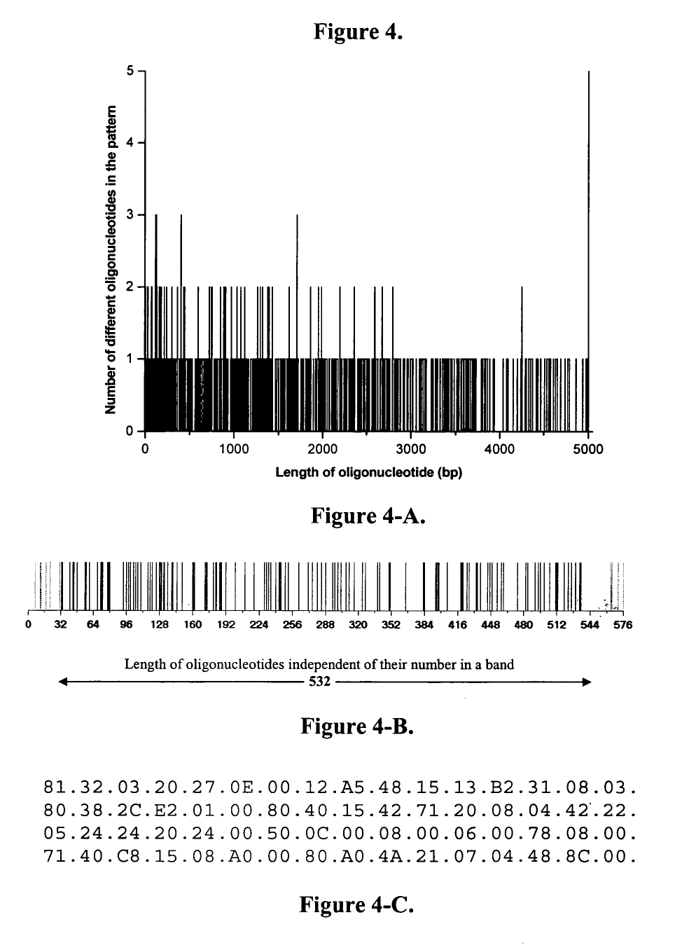

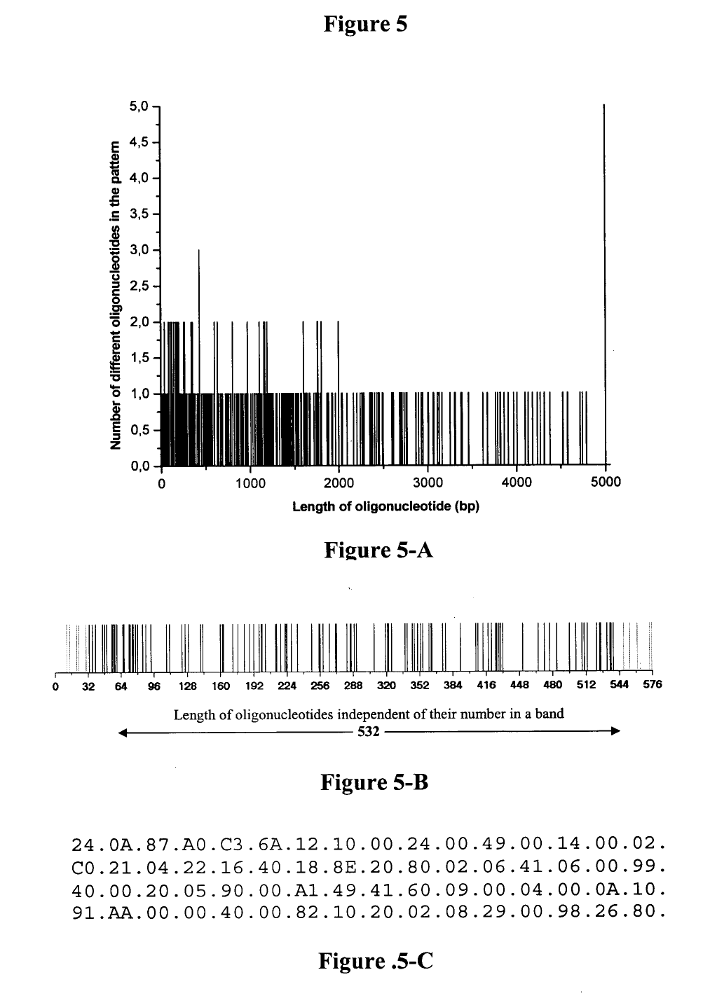

Illustrations in FIGS. 1A-1C, 2A-2C, 3A-3C, 4A-4C, 5A-5C demonstrate the different stages of the DC calculations. FIGS. “A” show the patterns of separation of crude digestion mixtures consisting of a wide range of oligonucleotides having the length from 1 to 5000 b.p. Axis Y indicates the number of different nucleotides in one pattern (having the same length) and bringing the character of insufficient information. FIGS. “B” demonstrate more formalized oligonucleotides pattern of separation (the range from 1 to 576 b.p.) in analog form (gif or another type of computer image). FIGS. “C” are presented as analog data of computer images in compact hexadecimal form.

### EXAMPLE 3

Example 3 demonstrates differences between closely related strains of Clamydophyla pneumoniae. Experiments were performed by methods of computer imitation on the basis of data of complete genome sequences [refs. 18, 19, 20]. As in Example 2, all the data were calculated with using the computer program “Silicone tube v.2.4”. C. pneumoniae strains were isolated in a context of a hospital infection, and had very similar cultural and biochemical properties and could not be differentiated without a profound study of genome structure or their effects on humans. The similarity of their genetic material is demonstrated in Table 3. The phylogenetical differences can be easily shown with the method of digital characterisation with restrictases BamHI and Bgl II.

The 512 byte DC of C. pneumoniae revealed with BamHI

for C. pneumoniae CWL029

01.01.00.00.00.80.00.00.00.00.00.00.00.24.14.41. 20.00.20.00.00.00.01.40.10.00.10.00.10.C0.00.80. 00.A0.00.00.04.40.00.00.48.40.40.00.00.00.10.02. 00.00.00.00.00.00.00.02.00.00.00.00.00.00.02.00

for C. pneumoniae AR39

01.01.00.00.00.80.00.00.00.00.00.00.00.20.14.41. 20.00.20.00.00.00.01.40.10.00.10.00.10.C0.00.80. 00.A0.00.00.04.40.00.00.48.02.00.00.00.00.10.02. 00.00.00.00.00.00.00.02.00.00.00.00.00.00.02.01

for C. pneumoniae J138

01.01.00.00.00.80.00.00.00.00.00.00.00.24.14.41. 20.00.20.00.00.00.01.40.10.00.10.00.10.C0.00.80. 00.A0.00.00.04.40.00.00.48.22.00.00.00.00.10.02. 00.00.00.00.00.00.00.02.00.00.00.00.00.00.02.00

The 512- byte DC of C. pneumoniae with Bgl II

for C. pneumoniae CWL029

0F.02.80.ED.20.29.64.C0.0E.82.42.47.12.10.84.2D. 14.69.44.80.A8.A3.09.84.03.32.40.2C.AA.68.20.14. 0C.80.04.02.63.10.A8.06.C5.50.8D.43.40.00.0A.44. 94.44.51.24.58.E1.07.00.00.42.20.2C.80.1A.A4.82

for C. pneumoniae AR39

0F.02.80.ED.20.29.64.C0.0E.82.42.47.12.10.84.2D. 14.69.44.80.A8.A3.09.84.03.32.40.2C.AA.68.20.14. 0C.80.04.82.63.10.A8.06.C5.70.8D.43.40.00.0A.44. 94.44.51.24.58.E2.07.00.00.42.20.2C.80.1A.A8.82

for C. pneumoniae J138

0F.02.80.ED.20.29.64.C0.0E.82.42.47.12.10.84.2D. 14.69.44.80.A8.A3.09.84.03.32.40.2C.A6.68.20.14. 0C.80.04.02.63.10.A8.06.C5.50.8D.43.40.00.0A.44. 94.44.51.24.58.E1.07.00.00.42.20.2C.80.1A.A4.82 The differences between the strains are emphasized by bold font.

### EXAMPLE 4

Demonstrates genetic similarities and differences between two strains of Mycobacterium tuberculosis [21,22].

DC values for both strains were obtained by the methods of computer simulation as described in Example 2. Data on the similarity of M. tuberculosis strains [21,22] are shown in Table 4.

### EXAMPLE 5

Example 5 demonstrates the process of DC calculations for the strain Mycoplasma pneumoniae M129 [29] having relatively short DNA. DC determination provided by the action of fourth restrictases: Agel, EcoRI, NheI, SpeI demonstrating relatively rare pattern of restriction with this type of DNA. All data were obtained with computer simulation with program “Silicone tube v2.4”.

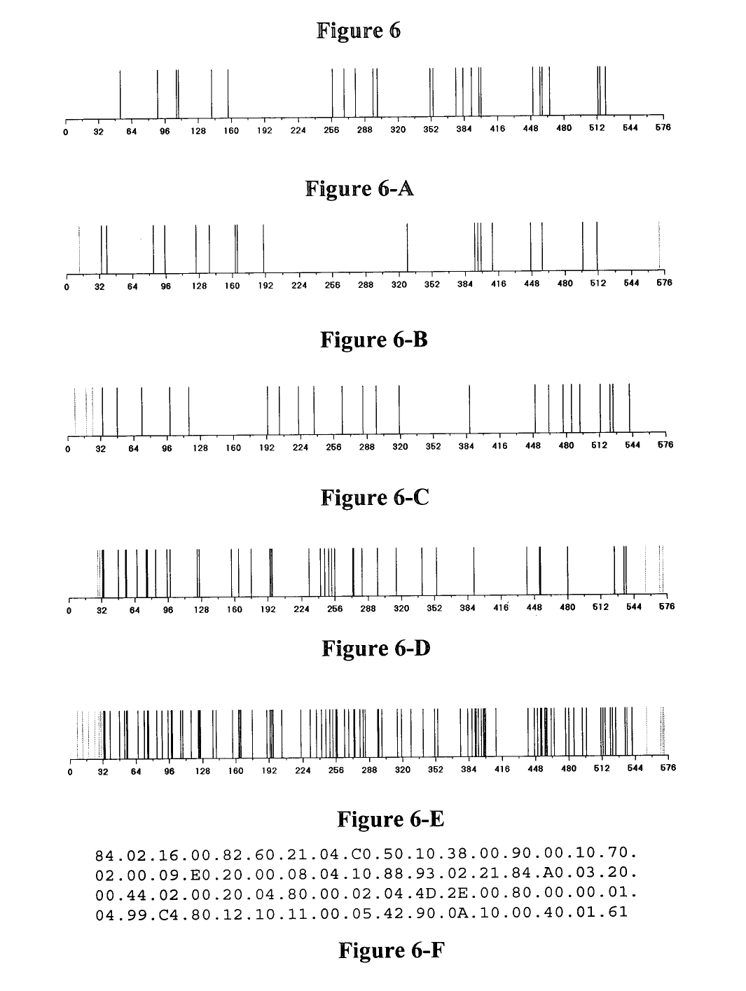

DNA from M. pneumoniae M129 was subjected to the action of said restrictases as separate probes. Products of restriction were separated as single products (FIGS. 6A-6D) and in the form of mixture (FIG. 6E). The 512-bite DC of M. pneumoniae M129 [29] in hexadecimal presentation obtained from analog data of FIG. 6-E is shown in FIG. 6F.

### EXAMPLE 6

Example 6 demonstrates the possibilities of determination of the differences and similarities between two strains of Helicobacter pylori belonging to different serotypes of one species. DC values for both strains were obtained by the methods of computer simulation as described in Example 2. Data on the similarity of Helicobacter strains [23, 24] in the form of DC is shown in Table 5.

### EXAMPLE 7

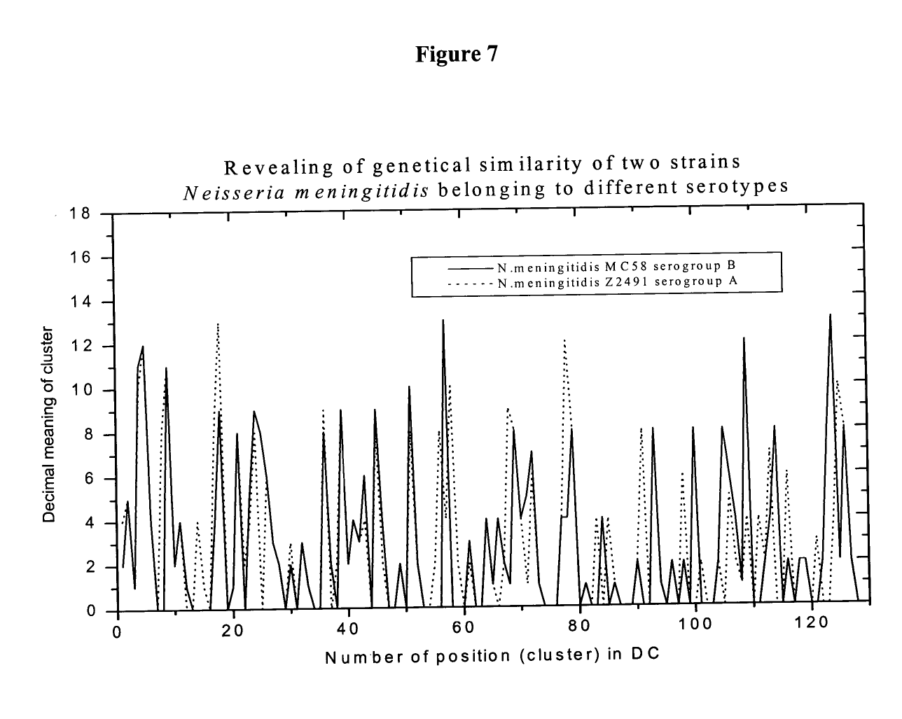

Demonstration of the similarity between two taxonomically remote strains of Neisseria meningitidis belonging to different serogroups [25, 26].The differences between two strains of N. meningitidis belonging to different serogroups A and B (strains Z2491 and MC58) are enough high, what is reflected in Table 6. It shows that both strains have only few identical numbers in DC. DCs shown as “decimal weight” of the hexadecimal number of DC vs. the position numbers, allows to illustrate the similarity more distinctly (FIG. 7).

### EXAMPLE 8

Example 8 demonstrates the possibility of transformation of the names of restrictases having hexanucleotide site of binding into the digital (hexadecimal) form.

There are only 4096 combinations characterizing the site of binding of all possible restrictases but having hexanucleotide palindrome of recognition. It is possible to fix the starting “word” AAAAAA characterizing the site of binding into the hexadecimal expression as 000, and the finish word as TTTTTT, (hex # FFF). The priority of order of sorting (changing in program counter) must be also fixed for example as A>C>G>T. According to this rule it is possible to calculate the row of all hexadecimal numbers corresponding to sites of binding having six nucleotides. These calculations were made with the program Silicone Tube v.2.4.4. According to them the intermediate meanings for combinations CCCCCC is 555 (hex), for GGGGGG is AAA (hex). The result of transformations is shown in Table 7. The position of the restriction inside of “word” is also determined as fourth hexadecimal digit on the next manner: ↓XXXY-XX-0 (hex), X↓XXXXX-1 (hex), XX↓XXXX-2 (hex). XXXXX↓X-6 (hex), . . . XXXXXX-7 (hex) (does not specify the case).

Novel hexadecimal names of known restrictases characterizing the mechanism of their actions are shown in the right column of Table 7.

### EXAMPLE 9

Determination of 512-byte DC for Saccharomyces cerevisiae. The yeast organism has complicated genomic structure consisting of 16 chromosomes and mitochondrial DNA [refs.30-31]. The possibility of determining DC for polychromosal genomes was also shown in the present invention. DC was determined with the restrictase Agel (total number of fragments in digestion mixture was 1543), for BspMII (total number of fragments 1331) and for Spl I (total number of fragments having different length 847). 512-byte DC of S. cerevisiae determined with AgeI:

A9.08.41.68.31.49.24.3A.01.C4.80.40.21.DE.01.10. C0.08.25.70.80.08.AC.71.22.11.52.0A.83.3B.3C.72. 28.8D.1B.E8.55.13.43.03.CB.1A.2C.40.98.34.AE.8B. 0B.2C.C9.B4.EA.10.20.90.2C.00.90.0A.7B.48.E4.00.

512-byte DC of S. cerevisiae determined with BspMII:

CC.FA.FF.5A.C1.CB.BC.FB.BE.A2.DB.E9.EE.FB.3B.47. 9E.7F.66.FB.F3.BF.FF.BD.EE.AF.BF.37.DE.FF.32.4C. EF.FD.DF.7F.B7.CF.FD.A3.D6.D8.5E.71.2C.04.35.B9. F3.6C.FE.8E.71.F5.D7.6F.70.7C.76.E5.82.16.A3.BF

512-byte DC of S. cerevisiae determined with Spl I:

AC.F7.4B.25.BF.C0.38.07.8B.36.94.0C.26.5D.8C.EC. 98.1A.D4.0E.4F.68.D2.02.F5.00.B4.2F.71.08.B2.83. 61.45.9A.C1.80.8F.42.30.41.12.A4.FE.D2.78.EC.78. 12.01.91.AE.90.9F.21.F6.39.06.28.6D.02.9A.A9.B0

TABLE 1
Characterization of DNA primary structure of two Rizobium
strains of in form of 128-bite hexadecimal passports.

| | | The number of bp in corresponding pattern and information | | | | | | | | | | | | | | | |
|---|---|---|---|---|---|---|---|---|---|---|---|---|---|---|---|---|---|
| | | about the presence of this pattern in the mixture of DNA | | | | | | | | | | | | | | | |
| | Endonuclease of | digestion. (1 - if the band is presents, 0- if the band of | | | | | | | | | | | | | | | |
| | restriction used | corresponding length is absents in the digestion's | | | | | | | | | | | | | | | |
| Strain of | for | mixture) | | | | | | | | | | | | | | | |
| Rizobium | characterization | 33 | 34 | 35 | 36 | 37 | 38 | 39 | 40 | 41 | 42 | 43 | 44 | 45 | 46 | 47 | 48 |
| 702 | Hind III | 0 | 0 | 0 | 1 | 1 | 1 | 0 | 0 | 1 | 1 | 0 | 0 | 0 | 1 | 1 | 0 |
| Hexadecimal | | 1C | | | | | | | | C6 | | | | | | | |
| expression | | | | | | | | | | | | | | | | | |
| 702 | Eco RI | 0 | 0 | 1 | 0 | 1 | 0 | 0 | 0 | 1 | 0 | 0 | 1 | 0 | 0 | 0 | 0 |
| Hexadecimal | | 28 | | | | | | | | 90 | | | | | | | |
| expression | | | | | | | | | | | | | | | | | |
| 703 | Hind III | 0 | 0 | 0 | 0 | 0 | 1 | 0 | 0 | 1 | 0 | 0 | 0 | 0 | 0 | 0 | 0 |
| Hexadecimal | | 04 | | | | | | | | 80 | | | | | | | |
| expression | | | | | | | | | | | | | | | | | |
| 703 | Eco RI | 0 | 1 | 1 | 0 | 0 | 1 | 0 | 0 | 1 | 0 | 0 | 1 | 0 | 0 | 0 | 1 |

| | | |
|---|---|---|
| Hexadecimal | 64. | 91 |
| expression | | |

| | | The number of bp in corresponding pattern and information | | | | | | | | |
|---|---|---|---|---|---|---|---|---|---|---|
| | | about the presence of this pattern in the mixture of DNA | | | | | | | | |
| | Endonuclease of | digestion. (1 - if the band is presents, 0- if the band of | | | | | | | | |
| | restriction used | corresponding length is absents in the digestion's | | | | | | | | |
| Strain of | for | mixture) | | | | | | | | |
| Rizobium | characterization | 49-145 | 146 | 147 | 148 | 149 | 150 | 151 | 152 | 153 |
| 702 | Hind III | . . .-. . . | 0 | 0 | 1 | 0 | 0 | 1 | 1 | 0 |
| Hexadecimal | | 52.1A.25. | 26 | | | | | | | |
| expression | | 4E.74.A8. | | | | | | | | |
| | | D2.9E.A2. | | | | | | | | |
| | | 24.70.B3 | | | | | | | | |
| 702 | Eco RI | . . .-. . . | 0 | 0 | 1 | 0 | 0 | 0 | 1 | 0 |
| Hexadecimal | | 9D.65.5A. | 02 | | | | | | | |
| expression | | E8D3.0A | | | | | | | | |
| | | 54.E9.5F. | | | | | | | | |
| | | 2E.BD.24 | | | | | | | | |
| 703 | Hind III | . . .-. . . | 0 | 1 | 0 | 0 | 0 | 1 | 1 | 1 |
| Hexadecimal | | 4E.2D.64. | 47 | | | | | | | |
| expression | | A2.0E.01. | | | | | | | | |
| | | A2.44.48. | | | | | | | | |
| | | 8C.90.4D. | | | | | | | | |
| 703 | Eco RI | . . .-. . . | 0 | 0 | 0 | 1 | 0 | 0 | 1 | 0 |

| | | |
|---|---|---|
| Hexadecimal | 97.14.42. | 12 |
| expression | 04.C4.91. | |
| | 1A.22.44. | |
| | 00.88.C8. | |

| | | |
|---|---|---|
| | | The number of bp in corresponding pattern and information |
| | | about the presence of this pattern in the mixture of DNA |
| | Endonuclease of | digestion. (1 - if the band is presents, 0- if the band of |
| | restriction used | corresponding length is absents in the digestion's |
| Strain of | for | mixture) |

| Rizobium | characterization | 154 | 155 | 156 | 157 | 158 | 159 | 160 | 161 |
|---|---|---|---|---|---|---|---|---|---|
| 702 | Hind III | 0 | 0 | 1 | 0 | 0 | 0 | 1 | 0 |
| Hexadecimal | | 22 | | | | | | | |
| expression | | | | | | | | | |
| 702 | Eco RI | 0 | 1 | 1 | 0 | 0 | 0 | 1 | 0 |
| Hexadecima | | 1F | | | | | | | |
| expression | | | | | | | | | |
| 703 | Hind III | 0 | 0 | 1 | 0 | 0 | 0 | 1 | 0 |
| Hexadecimal | | 22 | | | | | | | |
| expression | | | | | | | | | |
| 703 | Eco RI | 0 | 0 | 1 | 0 | 0 | 1 | 1 | 0 |

| | |
|---|---|
| Hexadecimal | 26 |
| expression | |

TABLE 2
Basic properties of total DNA of different bacteria used for
computer simulated experiments described in example 2.

| | | | | Number of | |
|---|---|---|---|---|---|
| | Name of | Length of | | cuttings/ | # of |
| ## | organism | DNA b.p. | Restrictase | genome | Fig. |
| 1 | Bacillus subtilis | 4.212.814 | PciI | 737 | 1a-c |
| | [16] | | | | |
| 2 | Esherichia coli | 5.529.376 | MfeI | 1412 | 2a-c |
| | 0157 [17] | | | | |
| 3 | Neisseria | 2.272.351 | Xma III | 723 | 3a-c |
| | meningitidis | | | | |
| | MC58 [25] | | | | |
| 4 | Aeropyrum pernix | 1.669.695 | Xma I | 729 | 4a-c |
| | K1 [27] | | | | |
| 5 | Mycoplasma | 580.074 | Hind III | 398 | 5a-c |
| | genitalium G37 | | | | |
| | [28] | | | | |

TABLE 3
Data on the similarity of full genomic DNA structure for the strains
of Clamydopyla pneumoniae CWL029, AR39 and J138 [18-20].

| | | Common number of nucleotides in Genomes | | | | |
|---|---|---|---|---|---|---|
| | | of Clamydopyla pneumoniae | | | | |
| | Names of | A + G + | | | | |
| | the Strains | C + T | A | G | C | T |
| 1 | CWL029 | 1230230 | 367242 | 249244 | 249955 | 363789 |
| 2 | AR39 | 1229858 | 363689 | 249834 | 249149 | 367112 |
| 3 | J138 | 1228267 | 366750 | 248819 | 248617 | 363080 |

TABLE 4
The differences of 2 Mycobacterium tuberculosis strains
revealed in their 512-bite digital characteristics
calculated in computer simulated experiments.

| Endonuclease | Digital codes (hex) of bacterial strains | |
|---|---|---|
| of | Mycobacterium tuberculosis | |
| restriction | Strain [21] | Strain CDC 1551 [22] |
| | 44.0A.26.B4.8E.69.51.A2. | 44.0A.26.B4.8E.69.51.A2. |
| BspMII | 0C.04.E7.D2.41.01.01.20. | 0C.04.67.D2.41.01.81.20. |
| | 30.E5.02.28.6E.60.4F.50. | 30.A5.02.29.66.40.4F.50. |
| | 43.0B.1C.3C.02.F9.01.C9. | 43.0B.1C.3C.02.F9.01.C9. |
| | 20.B0.82.6C.20.12.C0.00. | 20.B0.82.6C.20.12.C0.00. |
| | 00.C3.04.48.80.A4.10.82. | 00.C3.05.48.80.A4.50.82. |
| | F0.80.A0.02.88.23.80.00. | F0.80.A0.22.88.23.80.00. |
| | 02.00.75.01.E1.10.10.C0 | 02.00.54.01.E0.90.10.C0 |
| Cla I | F4.10.C1.21.02.CD.3C.15. | F4.10.C1.21.02.CD.3C.14. |
| | 00.44.02.30.62.13.42.15. | 00.44.02.30.42.13.42.15. |
| | 91.00.24.18.48.21.B2.4B. | D1.00.20.18.48.21.B2.4B. |
| | 83.70.09.F0.87.18.07.12. | 83.70.09.F0.87.18.07.12. |
| | 01.E0.32.C2.0C.51.0C.07. | 01.E0.33.C2.0C.51.0C.07. |
| | C2.B5.33.C3.78.95.14.05. | C2.95.33.43.78.95.14.05. |
| | 64.10.6B.14.03.45.D0.A0. | E4.10.6B.14.03.45.D0.A0. |
| | 00.88.04.09.94.8C.05.00 | 00.88.04.49.9C.8C.05.00 |

The differences between DC of both strains emphasized by bold fonts

TABLE 5
512-bite Digital codes (hex) of two strains of Helicobacter pylori

| | |
|---|---|
| Endonuclease | Digital codes of bacterial strains |
| of | Helicobacter pylori |

| restriction | Strain 26695 [23] | Strain J99 [24] |
|---|---|---|
| BspMII | FA.FF.BF.F3.AD.AB.FE.DB. | F2.7F.FB.F3.8D.6F.FA.EF. |
| | 77.FC.FF.BF.FE.BD.FB.E0. | 2F.F4.FC.BE.BF.F4.BD.E4. |
| | D5.56.10.BB.7D.3D.FB.5A. | F7.FC.A6.FB.5F.2F.FB.76. |
| | 57.EB.D2.A7.83.65.97.BF. | E7.08.B0.73.B3.65.EF.B7. |
| | AB.96.E5.35.FF.FA.68.DD. | EB.9E.DE.D6.39.DE.71.9A. |
| | 0D.E7.5D.F0.EF.94.FA.7D. | 2D.A9.BB.F7.E6.AE.FA.D7. |
| | 77.EE.FF.CC.C3.D5.49.1C. | 7D.D3.BF.FD.DD.91.7A.7A. |
| | EA.4D.BD.D6.B6.69.F9.4A | E6.03.F4.FC.06.CC.3A.6D |
| Mfe I | 00.10.00.88.28.00.02.10. | 00.00.00.C4.09.00.02.10. |
| | 10.00.00.00.60.00.00.00. | 00.80.00.00.00.00.00.00. |
| | 00.00.08.10.00.03.01.00. | 00.0A.A0.10.22.01.00.00. |
| | 0A.08.08.04.00.08.A4.00. | 0A.08.08.00.00.08.00.12. |
| | 00.00.80.00.00.80.05.00. | 01.20.00.01.00.10.00.00. |
| | 00.04.84.00.00.00.10.21. | 00.04.00.40.00.10.00.01. |
| | 00.00.02.00.10.00.00.00. | 00.40.10.00.00.00.00.00. |
| | 00.00.06.00.00.10.42.02 | 10.01.02.00.00.08.40.00 |

The differences in DC of different strains are emphasized by bold font

TABLE 6
Common and differences of 2 Neisseria meningitidis
strains revealed in their 512-bite DC (hex)

Endonuclease

| of | Digital codes of bacterial strains | |
|---|---|---|
| restriction | Strain Z2491 [26] | Strain MC58 [25] |
| XmaIII | 45.1A.C4.08.B2.40.04.10. | 25.1B.C4.00.B2.41.00.00. |
| | 8D.01.82.48.06.32.03.03. | 49.01.80.59.86.32.02.03. |
| | 10.09.02.92.43.40.82.00. | 10.08.20.92.43.60.94.00. |
| | 20.82.00.28.4A.20.20.04. | 20.A2.00.00.D0.00.30.04. |
| | 10.29.84.16.10.00.4C.80. | 14.21.84.57.10.00.44.80. |
| | 10.40.41.00.02.80.81.02. | 10.04.01.00.02.00.81.02. |
| | 06.00.20.02.05.21.40.42. | 02.08.00.02.86.41.C0.02. |
| | 70.06.02.20.30.04.A8.20 | 48.02.02.20.02.8D.28.20 |

The differences in DC emphasized by bold fonts

TABLE 7
Trivial names of restrictases having and their digital representation

| | | | | | New |
|---|---|---|---|---|---|
| | Trivial name | | Type of | Type of | Restrictases |
| | of | Site of | binding | cutting | “name” in |
| | restrictase | restriction | (in hex) | (in hex) | (hex) codes |
| 1 | Hind III | A\hat{ }AGCTT | 09F | 2 | 09F2 |
| 2 | Pci I | A\hat{ }CATGT | 13B | 2 | 13B2 |
| 3 | Age I | A\hat{ }CCGGT | 16B | 2 | 16B2 |
| 4 | Mlu I | A\hat{ }CGCGT | 19B | 2 | 19B2 |
| 5 | Spe I | A\hat{ }CTAGT | 1CB | 2 | 1CB2 |
| 6 | Bgl II | A\hat{ }GATCT | 237 | 2 | 2372 |
| 7 | Cla I | AT\hat{ }CGAT | 363 | 3 | 3633 |
| 8 | Vsp I | AT\hat{ }TAAT | 3C3 | 3 | 3C33 |
| 9 | Mfe I | C\hat{ }AATTG | 43E | 2 | 43 E2 |
| 10 | Nco I | C\hat{ }CATGG | 53A | 2 | 53A2 |
| 11 | Xma I | C\hat{ }CCGGG | 56A | 2 | 56A2 |
| 12 | Avr II | C\hat{ }CTAGG | 5CA | 2 | 5CA2 |
| 13 | Xma III | C\hat{ }GGCCG | 696 | 2 | 6962 |
| 14 | Spl I | C\hat{ }GTACG | 6C6 | 2 | 6C62 |
| 15 | Xho I | C\hat{ }TCGAG | 762 | 2 | 7622 |
| 16 | Afl II | C\hat{ }TTAAG | 7C2 | 2 | 7C22 |
| 17 | EcoR I | G\hat{ }AATTC | 83D | 2 | 83 D2 |
| 18 | BseP I | G\hat{ }CGCGC | 999 | 2 | 9992 |
| 19 | Nhe I | G\hat{ }CTAGC | 9C9 | 2 | 9C92 |
| 20 | BamH I | G\hat{ }GATCC | A35 | 2 | A352 |
| 21 | Bsp120 I | G\hat{ }GGCCC | A95 | 2 | A952 |
| 22 | Asp7181 | G\hat{ }GTACC | AC5 | 2 | AC52 |
| | Acc65 I | | | | |
| 23 | Sal I | G\hat{ }TCGAC | B61 | 2 | B612 |
| 24 | ApaL I | G\hat{ }TGCAC | B91 | 2 | B912 |
| 25 | BspM II | T\hat{ }CCGGA | D68 | 2 | D682 |
| 26 | Xba I | T\hat{ }CTAGA | DC8 | 2 | DC82 |
| 27 | Bsp1407 I | T\hat{ }GTACA | EC4 | 2 | EC42 |

1) A method for identification and specification of a prokaryotic or eukaryotic organism, the method comprising the steps of:
a) isolation of total DNA from the cells of the organism;
b) digestion of said DNA with highly specific restriction endonuclease(s) to yield oligonucleotides;
c) separation of said oligonucleotides according to their sizes;
d) recording absence and presence in the range of measured oligonucleotide sizes and aiving to the absence of an oligonucleotide size an equal informational value as to the resence of an oligonucleotide size;
e) transforming information obtained in steps c) and d) into a digital form by arranging the information in the form of a table having at least one rows and columns, said columns, equaling to a fixed range of different sizes of the oligonucleotides
f) using said table to express separation pattern in a digital form;
h) exploiting said digital form of information for identifying and specifying genetic materials from the organisms,, and
i) optionally, adding into final digital information a prefix containing a code or codes of the restriction endonuclease(s) used for said digestion.

2. The method according to claim 1, consisting of determination of digital numbers characterizing the primary structure of genomic DNA of studied organisms and comprising the steps of:
a) isolation of total DNA from cells of a studied organism;
b) full digestion of said DNA with a single type of restriction endonuclease having hexanucleotide site of recognition and affecting the formation of not less than 50, and not more than 3000 oligonucleotide fragments in digestion mixture;
c) separation of said oligonucleotide fragments according to their sizes;
d) recording absence and presence of any oligonucleotide size in the range of measured oligonucleotide sizes and giving to the absence of an oligonucleotide size an equal informational value as to the presence of an oligonucleotide size;
e) transforming information, received in steps c) and d) into the digital form by:
i) preparing a working table containing 2 rows, and 128, 256, or 512 columns;
ii) marking the uppermost row of the table from left to right side with consecutive integers salternatively, from #33 to #160, from # 33 to 280, or from #33 to #544, the numbers equaling to the sizes of the oligonucleotide fragments;
iii) inserting into the lowermost row of the table, digits, ones, or nulls, depending on the presence, or absence of corresponding size of an oligonucleotide;
iv) transforning 128^{th}, 256^{th}, or 512^{th}-digital binary numbers, created from the contents of the lowermost row taken from the columns of working table having numbers from #1 to #128, from # 1 to #256, or from #1 to #512, into one hexadecimal number; and,
v) forming of a single hexadecimal digital presentation of identification of the DNA by combining 2-6 digit hexadecimal prefix characterizing the structure of restriction binding site, or the number of protocol, taking into account previously specified information on the restriction endonuclease used for the digestion of the DNA of thea studied organism and hexadecimal series of numbers, received in step e iv.

3. The method according to claim 1, the method consisting of determination of digital numbers specifically characterizing the primary structure of genomic DNA of a studied organism and comprising the following steps:
a) isolation of total DNA from the cells of a studied organism;
b) digestion of said DNA with a first and a second types of restriction endonucleases having a hexanucleotide site of recognition and effecting formation of no less than 50, and no more than 3000 oligonucleotide fragments in digestion mixture;
c) separation of oligonucleotide fragments accordingly to the size of their chains;
d) recording absence and presence of any oligonucleotide size in the range of measured oligonucleotide sizes and giving to the absence of an oligonucleotide size an equal informational value as to the presence of an oligonucleotide size;
e) transforming information received in steps c) and d) into digital from by:
i) by preparing a working table containing boxes with 3 rows and 128, 256, or 512 columns;
ii) filling the uppermost row of the working table, from left to right side, with consecutive integers from #33 to #160, from #33 to #280, or from #33 to #544, numbers showing the position of the oligonucleotides in the digestion mixture and at the same time having the same size as the number of the box in the working table;
iii) inserting, into the middle row of the working table, digits, ones, or nulls, depending on the presence or absence of the corresponding patterns having given size in the separation pattern obtained with the first type of restriction endonucleases;
iv) inserting into the lowermnost row of the working table digits, ones or nulls, depending on the presence or absence of the corresponding patterns having given size in the separation products obtained with the second type of restriction endonuclease;
v) transformation of 128^{th}, 256^{th}, or 512^{th} digital binary numbers, created from contents of the middle and the lowermost rows, taken from columns of working table, having numbers from #1 to #128, from # 1 to #256, or from #1 to #512 in two hexadecimal numbers;
vi) formation of a single hexadecimal digital identification of DNA by combining 2-6 digit hexadecimal prefix, or the number of protocol, taking into account previously specified information on restriction endonucleases used for the digestion of DNA from the studied organism, and two hexadecimal series of numbers.

4. The method according to claim 1, the method consisting of determination of digital numbers specifically characterizing the primary structure of genomic DNA of a studied organism and comprising the following steps:
a) isolation of total DNA from the cells of a studied organism;
b) digestion of said DNA with more than two types of restriction endonucleases having hexanucleotide site of recognition and affecting the formation of no less than 50, and no more than 3000 oligonucleotide fragments in digestion mixture;
c) separation of the oligonucleotide fragments according to the size of their oligonucleotide chains;
d) recording, absence and presence of any oligonucleotide size in th range of measured oligonucleotide sizes and giving to the absence of an oligonucleotide size an equal informational value as to the presence of an oligonucleotide size;
e) transforming analog mode of results, received by the separation step, into the digital form by:
i) preparing a working table containing rows the number of which correspond to number or types of restriction endonucleasee of and, 128, 256, or 512 columns;
ii) inserting into the uppermost row of the table, from left to right side, consecutive integers from #33 to #160, from # 33 to 280, or from #33 to #544, the numbers showing the position of oligonucleotides in the separation products, and having the same size, as the number of the box in the table;
iii) inserting into the middle rows of the table digits, ones, or nulls, depending on the presence or absence of corresponding patterns having given length in separation products obtained with the 1^{st}, 2^{nd}, 3^{rd}, 4^{th}, . . . restriction endonuclease;
iv) inserting into the lowermost row of the working table digits, ones or nulls, depending on the presence or absence of corresponding patterns having given size in the separation products obtained with the last restriction endonuclease;
v) transforming 128^{th}, 256^{th}, or 512^{th} digital binary numbers created from contents of middle and lowermost rows, taken from columns of the working table, having numbers from #1 to #128, from # 1 to #256, or from #1 to #512 into two hexadecimal numbers, the numbers equaling with the number of restrictases;
vi) formation of a single hexadecimal digital identification of DNA by combination of 2-6 digit hexadecimal prefix, or the number of protocol, bringing together previously specified information about restriction endonucleases used for the digestion of DNA of studied organism, and the hexadecimal set of numbers received in step e.v.

5. An analysis kit of an organism's DNA said kit comprising:
a) reagents for isolation of said DNA;
b) specified restriction endonuclease to digest said DNA to yield oligonucleotides,
c) instructions to separate said oligonucleotides according to their sizes; and
d) instructions for obtaining the results in digital form.

6. The method according to claim 1 wherein information is arranged in table manually.

7. The method according to claim 1 wherein information is arranged in table by a computer program.
